(19) United States
(12) Patent Application Publication
(10) Pub. No.: US 2025/0129113 A1
(43) Pub. Date: Apr. 24, 2025

(54) NEW GLYCOSIDE COMPOUND AND PREPARATION PROCESS THEREOF

(71) Applicant: SunIn Biotech Co., Ltd., Tainan City (TW)

(72) Inventors: Hsu-En LO, Tainan City (TW); Wei-Chih SU, Tainan City (TW); Chun-Hsien HUANG, Tainan City (TW); Wan-Chen CHUNG, Tainan City (TW)

(73) Assignee: SunIn Biotech Co., Ltd., Tainan City (TW)

(21) Appl. No.: 18/924,291

(22) Filed: Oct. 23, 2024

Related U.S. Application Data
(60) Provisional application No. 63/592,245, filed on Oct. 23, 2023.

(51) Int. Cl. C07H 15/18; A61K 31/7012; A61P 17/02

(52) U.S. Cl. CPC C07H 15/18; A61K 31/7012; A61P 17/02

(57) ABSTRACT

The present invention provides a new glycoside compound, which has a high efficacy in promotion of cell proliferation and wound healing. Also provided include a pharmaceutical composition comprising the compound and a method for treating a diabetic wound using the compound.

### CROSS REFERENCE

This Non-provisional application claims the priority under 35 U.S.C. § 119 (e) on U.S. Patent Provisional Application No. 63/592,245 filed on Oct. 23, 2023, the entire contents of which are hereby incorporated by reference.

### FIELD OF THE INVENTION

The present invention relates to a new glucoside compound, which has an efficacy in promoting the cell proliferation and wound healing.

### BACKGROUND OF THE INVENTION

Acteoside, also known as Verbascoside, is a caffeoyl phenylethanoid glycoside, [1] in which the phenylpropanoid caffeic acid and the phenylethanoid hydroxytyrosol from an ester and an ether bond respectively, to the rhamnose part of a disaccharide, namely β-(3′,4′-dihydroxyphenyl)ethyl-O-α-L-rhamnopyransyl(1→3) -β-D-(4-O-caffeoyl)-glucopyranoside. [2] Acteoside is a natural substance found in the plants, such as the species in the families of the order Lamiales (syn. Scrophulariales). [3] It is a natural substance which can be found in the plants, such Rehmannia glutinosa, Cistanche tubulosa, and the like. Acteoside can also be produced in plant cell cultures of Leucosceptrum sp (Lamiaceae) and Syringa sp (Oleaceae), [4] or in hairy roots cultures of Paulownia tomentosa (empress tree, Paulowniaceae). [5] However, it is difficult to chemically synthesize Acteoside. Therefore, the mass production of Acteoside for medicines is impossible.

These plant species containing Acteoside and their extracts have a long history of use treating disease in traditional Chinese medicine, and Acteoside broadly used and studied. It is known that Acteoside has antimicrobial activity, [6] against Staphylococcus aureus. [7] It can also have anti-inflammatory properties. [8] However, Acteoside (itself or in the form of extracts) has not been explored as a potential medicine or health food Supplement for promoting cell proliferation.

It is still desirable to develop new Acteoside derivates to provide better alternatives.

### BRIEF SUMMARY OF THE INVENTION

Accordingly, the present invention provides a new glycoside compound, which can be chemically synthesized.

In one aspect, the present invention provides a new compound, named as Nusynside, which has the following structure:

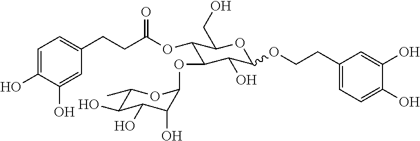 [SMILES: CC1OC(OC2C(O)C(OCCc3ccc(O)c(O)c3)OC(CO)C2OC(=O)CCc2ccc(O)c(O)c2)C(O)C(O)C1O]

In one example of the invention, Nusynside has an efficacy in improving cell proliferation.

In one example of the invention, Nusynside has an efficacy in reducing cellular inflammatory responses through downregulating the expression of TNF-α and IL-6 genes.

In one particular example of the invention, Nusynside is ascertained to have a good efficacy in treating a diabetic wound such as diabetic foot ulcer (DFU) and impaired oral wound of a diabetic subject.

In another aspect, the present invention provides a composition/pharmaceutical composition for wound healing comprising a therapeutically effective amount of Nusynside, and a pharmaceutically acceptable carrier.

In a yet aspect, the present invention provides a method for promoting cell proliferation in a subject, comprising administering to the subject Nusynside in a therapeutically effective amount or the composition/pharmaceutical composition thereof.

In a further yet aspect, the present invention provides a use of Nusynside for manufacturing a medicament for cell proliferation and wound healing.

In a further yet aspect, the present invention provides a use of Nusynside for manufacturing a medicament for treating a diabetic wound.

In one embodiment of the present invention, the diabetic wound is diabetic foot ulcer (DFU) or impaired oral wound of a diabetic subject.

In a yet further aspect, the present invention provides a process for preparing Nusynside comprising the steps of:
reacting Acteoside as starting material in a catalytic amount of Pd/C in autoclave, which is purged with hydrogen several times to obtain a crude product;
stirring the crude product in hydrogen to the completion of reaction to obtain a mixture; and
filtering and purifying the mixture and collecting a final product.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The drawings presenting the preferred embodiments of the present invention are aimed at explaining the present invention. It should be understood that the present invention is not limited to the preferred embodiments shown.

FIG. 1 provides the results of the Molecular Weight (MW) Detection for Nusynside.

FIG. 2 provides the results of the NMR character detection for Nusynside.

FIG. 3 provides the result of the purity detection for Nusynside by HPLC C18 Column at 280 nm, showing that the purity was more than 97%.

FIG. 4 shows the efficacy of Nusynside in promoting the proliferation of HaCaT cells cultured with 25 mM glucose for 48 hours, including the results of the control group (Control), and experimental groups: Nusynside 3.2 μM, Nusynside 16 μM, and Nusynside 32 μM (Results are expressed as mean+SD, ** p<0.01; p<0.001).

FIG. 5 shows the efficacy of Nusynside in promoting the proliferation of Hs68 cells cultured with 25 mM glucose for 24 hours, including the results of the control group (Control), and experimental groups: Nusynside 3.2 μM, Nusynside 16 μM, and Nusynside 32 μM (Results are expressed as mean+SD, ** p<0.01; p<0.001).

FIGS. 6A-6B are histograms of pro-inflammatory gene expression analyzed by real-time PCR method. FIG. 6A shows the gene expression fold of TNF-α; FIG. 6B shows the gene expression fold of IL-6. (Results are expressed as mean+SD, ** p<0.01; **** p<0.0001).

FIG. 7 shows the wound healing rate of Nusynside test group (** p<0.01; **** p<0.0001).

FIG. 8 shows the skin wound images of Nusynside test group comparing to the Positive Control group.

### DETAILED DESCRIPTION OF THE INVENTION

Unless otherwise defined herein, scientific and technical terms used herein have the meanings that are commonly understood by those of ordinary skill in the art.

As used herein, the singular forms “a”, “an”, and “the” include plural referents unless the context clearly dictates otherwise. Thus, for example, reference to “a sample” includes a plurality of such samples and equivalents thereto known to those skilled in the art.

The present invention provides a new compound, named as Nusynside, which has the following structure:

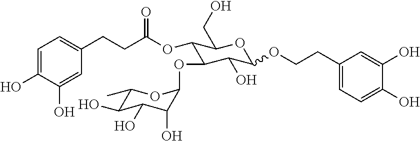 [SMILES: CC1OC(OC2C(O)C(OCCc3ccc(O)c(O)c3)OC(CO)C2OC(=O)CCc2ccc(O)c(O)c2)C(O)C(O)C1O]

According to the invention, Nusynside may be prepared by using Acteoside as starting material, see below.

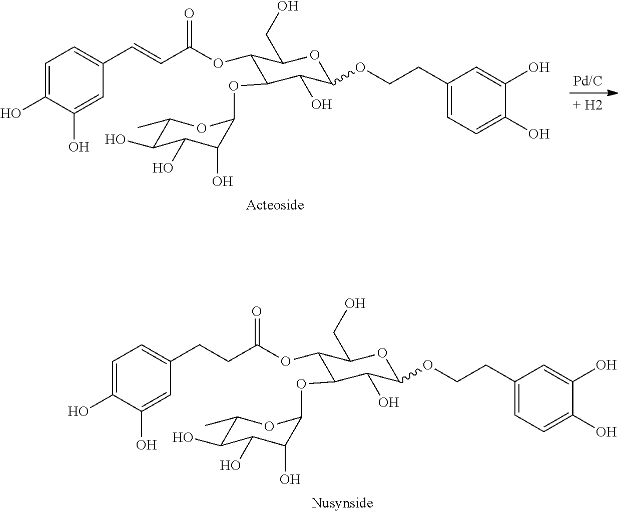 [SMILES: CC1OC(OC2C(O)C(OCCc3ccc(O)c(O)c3)OC(CO)C2OC(=O)/C=C/c2ccc(O)c(O)c2)C(O)C(O)C1O ; CC1OC(OC2C(O)C(OCCc3ccc(O)c(O)c3)OC(CO)C2OC(=O)CCc2ccc(O)c(O)c2)C(O)C(O)C1O]

For example, Nusynside is obtained by the method of the following steps:
reacting Acteoside as starting material in a catalytic amount of Pd/C in autoclave, which is purged with hydrogen several times to obtain a crude product;
stirring the crude product in hydrogen to the completion of reaction to obtain a mixture;
filtering and purifying the mixture and collecting a final product.

As used herein, the term “subject” refers to a human or an animal, including a human or an animal. In the present invention, the subject or patient is a human with a diabetic wound such as diabetic foot ulcer (DFU) or oral wound of a diabetic subject.

The term “pharmaceutically acceptable carrier” used herein is intended to include any and all solvents, dispersion media, coatings, isotonic and absorption delaying agents, and inert ingredients. The use of such pharmaceutically acceptable carriers for active pharmaceutical ingredients is well known in the art. The term also refers to a carrier(s), diluent(s) or excipient(s) that is acceptable, in the sense of being compatible with the other ingredients of the formulation and not deleterious to the subject to be administered with the composition/pharmaceutical composition. Any carrier, diluent or excipient commonly known or used in the field may be used in the invention, depending to the requirements of the pharmaceutical formulation. Said carrier may be a diluent, vehicle, excipient, or matrix to the active ingredient. Some examples of appropriate excipients include lactose, dextrose, sucrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates; sweeteners; and flavoring agents. According to the present invention, a particular example of the carrier is an ointment NuDFC7.

As used herein, the term “treat,” “treating,” or “treatment” refers to both therapeutic treatment and prophylactic or preventative measures, wherein the object is to prevent, slow down, and/or halt the development of wound or DFU. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable.

The term “therapeutically effective amount” as used herein refers to an amount of a pharmaceutical agent which, as compared to a corresponding subject who has not received such amount, results in an effect in treatment, healing, prevention, or amelioration of a disease, disorder, or side effect, or a decrease in the rate of advancement of a disease or disorder. The term also includes within its scope amounts effective to enhance normal physiological function. For use in therapy, the therapeutically effective amount of the compound is formulated as a pharmaceutical composition for administration. Accordingly, the invention further provides a pharmaceutical composition comprising a therapeutically effective amount of the preparation of Nusynside, and one or more pharmaceutically acceptable carriers.

According to the invention, Nusynside is confirmed to have an efficacy of wound healing, anti-inflammation, angiogenesis, and extracellular matrix modulation in high concentration glucose-treated human keratinocytes and Human fibroblast. It is ascertained that Nusynside possessed the greatest potential to restore the impairment of keratinocytes and fibroblast caused by high glucose concentrations. It was found in the present invention that Nusynside downregulated the gene expression of TNF-α and IL-6. Collectively, these data support the potential of Nusynside for therapeutic development in the treatment of diabetic foot ulcer (DFU). [9] [10]

It is also ascertained in the invention that Nusynside has an efficacy in the improvement of cell viability and wound healing capacity of epithelial cells under high glucose condition. It was unexpectedly found in the invention that Nusynside attenuated the high glucose-induced cytotoxicity and impaired healing by downregulation of oxidative stress. The results demonstrated that Nusynside increased the activity of the antioxidant enzyme SOD and reduced the oxidative stress indicator as well as apoptosis. Accordingly, Nusynside may be beneficial to treat impaired oral wound healing for diabetic patients.

The present invention is further illustrated by the following examples, which are provided for the purpose of demonstration rather than limitation.

### Examples

### Example 1. Preparation of Nusynside

The magnet stirrer, starting material Acteoside (50 mg), and a catalytic amount of 10% Pd/C was charged in autoclave at room temperature in methanol. The reactor was purged with hydrogen several times. Then the reaction was stirred in hydrogen with 200 psi pressure over night. The mixture was checked with HPLC. After the reaction was completed, the mixture was filtered with a pad of celite. The filtrate was concentrated to give a mixture. The mixture was purified with C18 gel and collected to give a brown solid (40 mg, 97% HPLC purity) (reduced Acteoside, C03107).

The product was named as Nusynside, and was analyzed, and characterized.

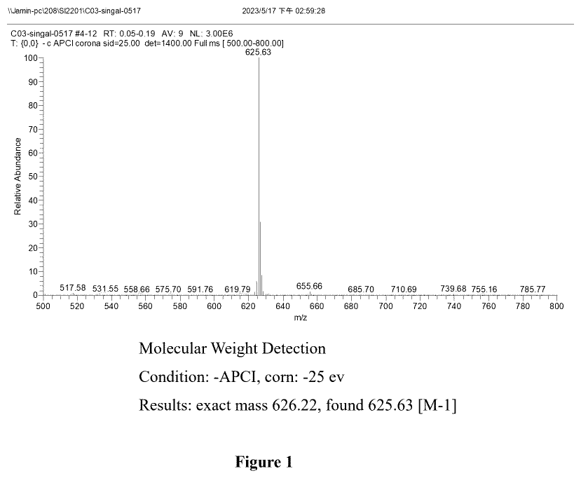

The condition and results of the molecular weight Detection were given below, as shown in FIG. 1:
Condition: -APCI, corn: −25 ev
Results: exact mass 626.22, found 625.63 [M−1].

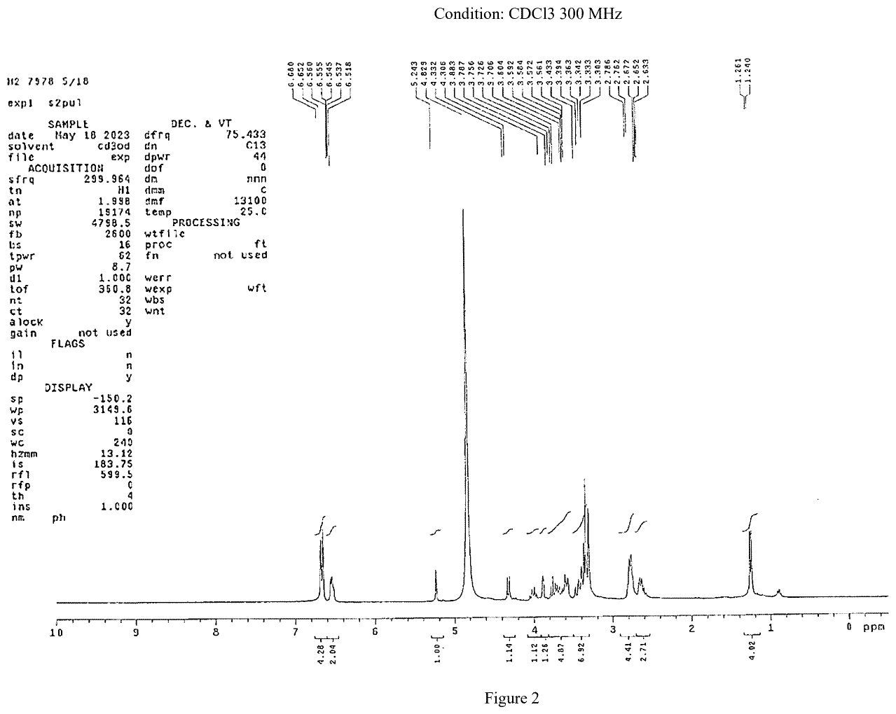

The results of the NMR character detection in CDCl3 300 MHz were shown in FIG. 2.

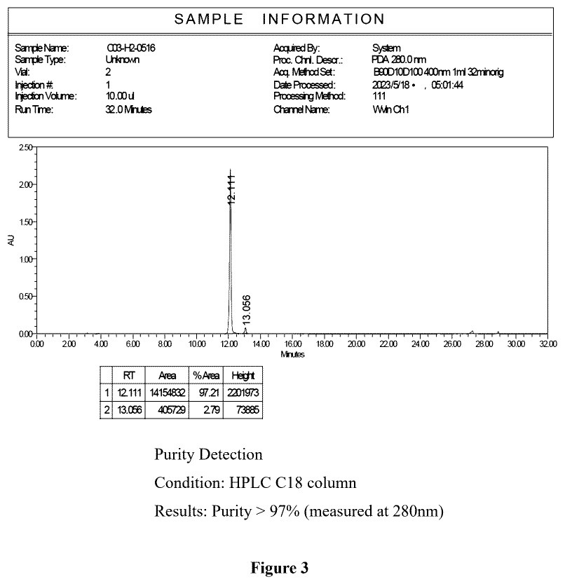

The end product, Nusynside, was also analyzed by HPLC C18 column, showing that the purity measured at 280 nm was more than 97%, as shown in FIG. 3.

The 1H NMR was performed and the results of the analysis are given below: ¹HNMR (CD3OD 300 MHz) δ 6.66 (d, J=8.4 Hz, 4 H, ArH), 6.60-6.50 (m, 2H, ArH), 5.24 (s, 1H), 1.14 (d, J=7.8 Hz, 1H), 4.10-3.20 (m, 12H), 2.90-2.50 (m, 6H, benzylic and alpha position), 1.25 (d, J=6.3 Hz, 3 H, CH3), Mass-APCI exact mass 626.22, found 625.63 [M−1].

### Example 2. Efficacy Tests

### Cell Culture

Human Foreskin Fibroblast, Hs68 cells and human keratinocytes, HaCaT cells were cultured in Dulbecco's Modified Eagle Medium (DMEM) added with glucose and 10% fetal calf serum, and then the cells were put in a humidified incubator with an air of 5% carbon dioxide and 95% oxygen. The medium was changed every two days. The subculture was performed, and then treated with 0.25% trypsin (TRYPSIN-EDTA) mixture. Finally, the cells were collected and diluted at a ratio of 1:5 for re-culture. The cells used in the efficacy test had passage numbers ranging from 12 to 40.

Human acute monocytic leukemia, THP1 cells were cultured in Roswell Park Memorial Institute (RPMI) 1640 medium added with 10% fetal calf serum, and then the cells were put in a humidified incubator at 37° C., and with an air of 5% carbon dioxide and 95% oxygen.

### Cell Proliferation Assay

HaCaT cells at the number of 5×10³ were cultured Culture in DMEM culture medium for 48 hours. The cell growth and survival status were analyzed with the CCK-8 reagent kit (DOJINDO MOLECULAR TECHNOLOGIES, KUMAMOTO, JAPAN). 10 μL CCK-8 solution was added to each cell culture and the cells were incubated at 37° C. for one hour, and then the cells were measured by the spectrophotometer MICROPLATE READER (THERMO LABSYSTEMS MULTISKAN RC) at 450 nm absorption, and the experimental results were analyzed by a comparison with the control group.

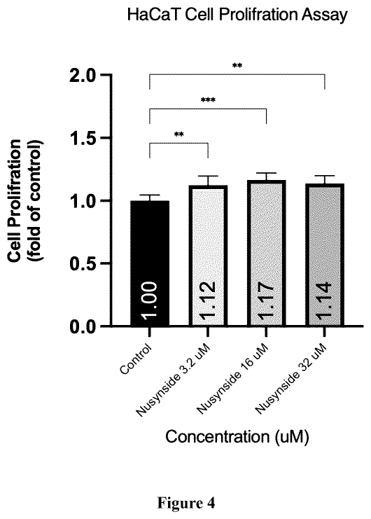

As shown in FIG. 4, Nusynside as chemically synthesized according to the invention promoted the cell proliferation of HaCaT cells by 12% at a concentration of 3.2 μM, 17% at a concentration of 16 μM, and 14% at a concentration of 32 μM.

The Hs68 cells were cultured in DMEM culture medium for 24 hours. The cell growth and survival status were analyzed with the CCK-8 reagent kit (DOJINDO MOLECULAR TECHNOLOGIES, KUMAMOTO, JAPAN). 10 μL CCK-8 solution to each cell culture was added and the cell culture was incubated at 37° C. for one hour, and then the cells were measured by the spectrophotometer MICROPLATE READER (THERMO LABSYSTEMS MULTISKAN RC) at 450 nm absorption, and the experimental results were analyzed by a comparison with the control group.

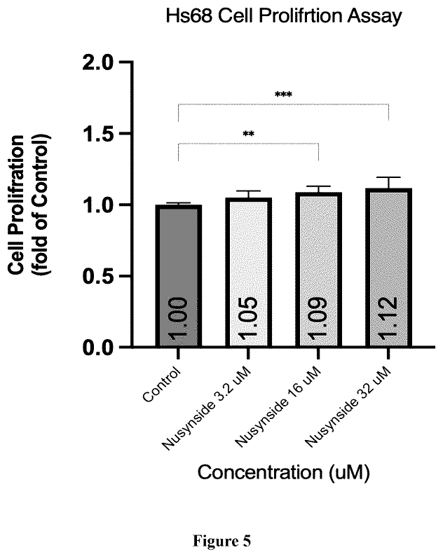

As shown in FIG. 5, the Nusynside as chemically synthesized according to the invention promoted the cell proliferation of Hs68 cells by 9% at a concentration of 16 μM, and 12% at a concentration of 32 μM.

### Real-Time PCR Inflammatory Gene Expression Assay

THP1 cells were treated with 100 ng/ml of phorbol 12-myristate 13-acetate (PMA) for 24 hours to induce activation. The following day, the medium was replaced with RPMI 1640 (25 mM Glucose, 10% FBS) and incubated for another 24 hours. The cells were then differentiated into M1 phase macrophages by switching to RPMI 1640 medium containing LPS (10 pg/ml) and IFN-γ (20 ng/ml), and into M2 phase macrophages using RPMI 1640 medium containing IL-4 (20 ng/ml) and IL-13 (20 ng/ml). During the 48-hour differentiation process, the test drug Nusynside (16 μM) was added. After 24 hours of differentiation, mRNA from M1 and M2 phase macrophages was extracted using the PROTECH Kit, and reverse transcription of mRNA into cDNA was performed with the Protech MMLV Reverse Transcription Kit. Gene expression was subsequently observed. According to the POWERTRACK SYBR dye protocol, the cDNA was mixed, and gene expression was analyzed using the StepOnePlus Real-Time PCR Systems to examine inflammatory gene expression when THP1 cells were differentiated into M1 and M2 phase macrophages in the presence of Nusynside (16 μM).

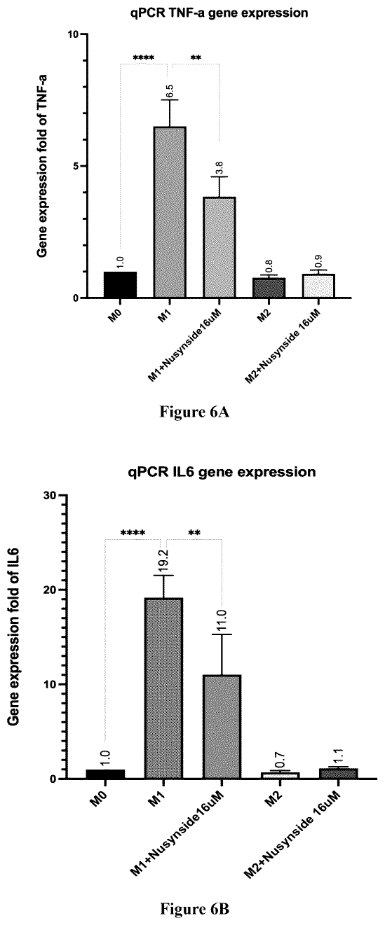

FIGS. 6A and 6B show that during the differentiation of THP1 cells into M1-type macrophages, the genes for pro-inflammatory tumor necrosis factor TNF-α and IL-6 were significantly upregulated. However, in the presence of 16 μM Nusynside, the expression of TNF-α and IL-6 genes was significantly downregulated during the differentiation of THP1 into M1-type macrophages. These results indicated that Nusynside at a concentration of 16 μM has effect in reducing cellular inflammatory responses.

### Diabetic Mice Wound Healing Study

BKS.Cg-Dock7m+/+ Leprdb/Jnarl male mice, an established diabetes model, were obtained from the National Applied Research Laboratories, Taiwan. The experiment was conducted when the mice were 6 to 7 weeks old. A full-skin wound of the same size with a diameter of about 6 mm was made on the left and right sides of each mouse's back.

Total 14 mice were randomly divided into 4 groups based on body weight: (i) Nusynside test group (administrated with Nusynside 0.23 mg) (n=4); (ii) Positive Control group (administrated with cream containing Centella Asiatica and Plectranthus amboinicus extract) (n=4); (iii) Vehicle Control group (administrated with ointment base) (n=3); (vi) Blank group (n=3). The animals underwent bilateral incisions and were housed individually in separate cages after the incisions, in the IVC housing system of the general SPF breeding area. Daily external cage observations were conducted by the animal caretakers. On the day of the incision and during follow-up photography, the body weight of the animals, wound appearance, and wound area were measured and recorded, except that the wound had healed resulting in it disable to be measured, the measurements were taken twice a week.

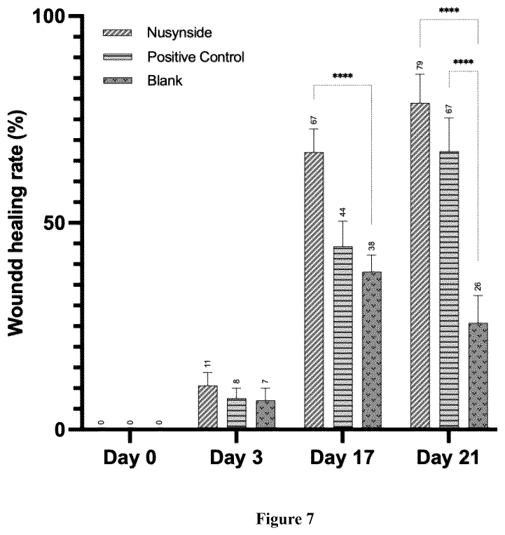

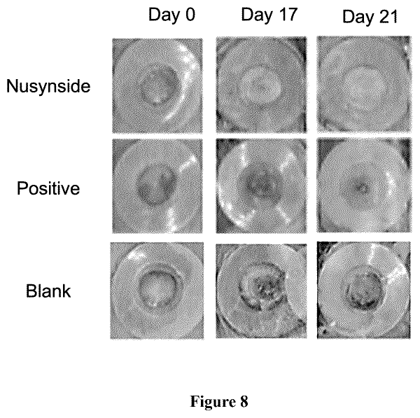

As shown in FIG. 7 and FIG. 8, it was found that Nusynside had extraordinary efficacy in wound healing. According to the result, the Nusynside (Day 17 after continuous administration) significantly increased the wound healing rate by comparing to the Blank group, while the Positive Control group showed no therapeutic effect until Day 21 after continuous administration. It is ascertained that the Nusynside provided a better efficacy in wound healing than Centella Asiatica and Plectranthus amboinicus extract cream which is a common commercial product used for treating DFU so far.

The result support the potential of Nusynside for therapeutic development in treating DFU or impaired oral wound of a diabetic subject.

While the present invention has been disclosed by way preferred embodiments, it is not intended to limit the present invention. Any person of ordinary skill in the art may, without departing from the spirit and scope of the present invention, shall be allowed to perform modification and embellishment. Therefore, the scope of protection of the present invention shall be governed by which defined by the claims attached subsequently.

### REFERENCE

1. Taskova, Rilka Mladenova; Gotfredsen, Charlotte Held; Jensen, Søren Rosendal (2005). “Chemotaxonomic markers in Digitalideae (Plantaginaceae)”. Phytochemistry. 66 (12): 1440-7.
2. Andary, C; Wylde, R; Laffite, C; Privat, G; Winternitz, F (1982). “Structures of verbascoside and orobanchoside, caffeic acid sugar esters from Orobanche rapum-genistae”. Phytochemistry. 21 (5): 1123-7.
3. Schlauer, Jan; Budzianowski, Jaromir; Kukułczanka, Krystyna; Ratajczak, Lidia (2011). “Acteoside and related phenylethanoid glycosides in Byblis liniflora Salisb. Plants propagated in vitro and its systematic significance”. Acta Societatis Botanicorum Poloniae. 73 (1): 9-15.
4. Inagaki, Nobuyuki; Nishimura, Hiroaki; Okada, Minoru; Mitsuhashi, Hiroshi (1991). “Verbascoside production by plant cell cultures”. Plant Cell Reports. 9 (9): 484-487.
5. Wysokiińska, H; Rózga, M (1998). “Establishment of transformed root cultures of Paulownia tomentosa for verbascoside production”. Journal of Plant Physiology. 152 (1): 78-83.
6. Pardo, F; Perich, F; Villarroel, L; Torres, R (1993). “Isolation of verbascoside, an antimicrobial constituent of Buddleja globosa leaves”. Journal of Ethnopharmacology. 39 (3): 221-2.
7. Guillermo Avila, José; De Liverant, Juliana G; MartíNez, Andrés; MartíNez, Gabriel; Muñoz, José Luis; Arciniegas, Amira; Romo De Vivar, Alfonso (1999). “Mode of action of Buddleja cordata verbascoside against Staphylococcus aureus”. Journal of Ethnopharmacology. 66 (1): 75-8.
8. Speranza, L.; Franceschelli, S.; Pesce, M.; Menghini, L.; Patruno, A.; Vinciguerra, I.; De Lutiis, M. A.; Felaco, M.; Felaco, P.; Grilli, A. (2009). “Anti-inflammatory properties of the plant Verbascum mallophorum”. Journal of Biological Regulators and Homeostatic Agents. 23 (3): 189-95.
9. Wound healing and anti-inflammatory activities induced by a Plantago australis hydroethanolic extract standardized in verbascoside. J Ethnopharmacol. 2018 Oct. 28; 225:178-188.
10. Polyherbal formulation exerts wound healing, anti-inflammatory, angiogenic and antimicrobial properties: Potential role in the treatment of diabetic foot ulcers. Saudi J Biol Sci. 2022 July; 29 (7): 103330.

1. A compound of Formula (I):

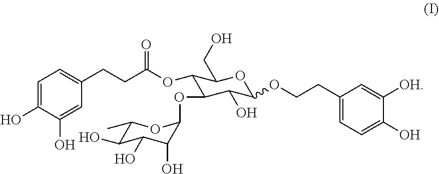 [SMILES: CC1OC(OC2C(O)C(OCCc3ccc(O)c(O)c3)OC(CO)C2OC(=O)CCc2ccc(O)c(O)c2)C(O)C(O)C1O]

2. The compound of claim 1, which is capable of improving cell proliferation.

3. The compound of claim 1, which is capable of reducing cellular inflammatory responses through downregulating the expression of TNF-α and IL-6 genes.

4. A pharmaceutical composition comprising a therapeutically effective amount of the compound as defined in claim 1, and a pharmaceutically acceptable carrier.

5. The pharmaceutical composition of claim 4, which is effective in wound healing and cell proliferation.

6. A composition for wound healing and cell proliferation comprising a therapeutically effective amount of the compound as defined in claim 1.

7. A method for promoting cell proliferation in a subject, comprising
administering to the subject (a) the compound as defined in claim 1 in a therapeutically effective amount or (b) a pharmaceutical composition comprising a therapeutically effective amount of the compound, and a pharmaceutically acceptable carrier.

8. A method for treating a diabetic wound in a subject, comprising
administering to the subject (a) the compound as defined in claim 1 in a therapeutically effective amount or (b) a pharmaceutical composition comprising a therapeutically effective amount of the compound, and a pharmaceutically acceptable carrier or (c) a composition suitable for wound healing and cell proliferation comprising a therapeutically effective amount of the compound.

9. The method of claim 8, wherein the diabetic wound is diabetic foot ulcer (DFU).

10. The method of claim 8, wherein the diabetic wound is impaired oral wound of a diabetic subject.

11. A process for preparing the compound as defined in claim 1, comprising the steps of:
reacting Acteoside as starting material in a catalytic amount of Pd/C in autoclave, which is purged with hydrogen several times to obtain a crude product;
stirring the crude product in hydrogen to the completion of reaction to obtain a mixture; and
filtering and purifying the mixture and collecting a final product.
